(19) United States
(12) Patent Application Publication
(10) Pub. No.: US 2024/0197583 A1
(43) Pub. Date: Jun. 20, 2024

(54) Hair Relaxer

(71) Applicant: BASF SE, Ludwigshafen (DE)

(72) Inventors: Tosin Dania, Lagos (NG); Marc Beuche, Levallois-Perret Cedex (FR); Ali Blessing, Lagos (NG); Igbinoba Osarenren, Lagos (NG)

(21) Appl. No.: 18/288,205

(22) PCT Filed: Apr. 25, 2022
(86) PCT No.: PCT/EP2022/060812

§ 371 Date: Oct. 25, 2023

(30) Foreign Application Priority Data
Apr. 30, 2021 (EP) 21171559.4

(51) Int. Cl. A61K 8/19; A61K 8/06; A61K 8/34; A61K 8/365; A61K 8/43; A61K 8/60; A61K 8/92; A61Q 5/04

(52) U.S. Cl. CPC A61K 8/19; A61K 8/062; A61K 8/34; A61K 8/342; A61K 8/365; A61K 8/43; A61K 8/602; A61K 8/922; A61Q 5/04

(57) ABSTRACT

This invention relates to a concentrated hair relaxer formulation, which is ready to use after mixing with water and which preferable is filled in 1-pack, wherein both, the concentrated and ready-to use hair relaxer formulation show improved stability properties and excellent smoothing results with less damage of hair. Furthermore, the invention relates to a method for producing such a concentrated hair relaxer formulation, a method for relaxing hair and a 1-pack containing such concentrated hair relaxer formulation and its use.

### FIELD OF THE INVENTION

This invention relates to a concentrated hair relaxer formulation, which is ready to use after mixing with water and which preferable is filled in 1-pack, wherein both, the concentrated and the ready to use hair relaxer formulation show improved stability properties and excellent smoothing results with less damage of hair. Furthermore, the invention relates to a method for producing such a concentrated hair relaxer formulation, a method for relaxing hair and a 1-pack containing such concentrated hair relaxer formulation and its use.

### PRIOR ART

There are many techniques and compositions for styling or altering the appearance of hair. In today's market, there is an increasing demand for hair products referred as to “hair relaxer” or “hair straightener” or “hair smoother” which can relax or straighten curly or kinky or wavy hair.

In general, compositions relaxing the hair comprising an alkaline composition such as inorganic metal hydroxides or organic hydroxides, which penetrate in the structure of the hair. Hair relaxing products comprising sodium hydroxide or potassium hydroxide as an alkaline composition are also called lye-based products. Hair relaxing products comprising other alkaline agents like lithium hydroxide or organic hydroxides like guanidine hydroxide as an active agent are called as no-lye products. Lye hair relaxer are extremely effective but have some skin-irritating properties. No-lye hair relaxer based on guanidine hydroxide are supposed to be less skin-irritating, but less stable as lye relaxer.

However, guanidine hydroxide as the active agent is in combination with inorganic hydroxides not very stable and therefore most commercial products are based on a two-package system: one package containing inorganic hydroxides such as Calcium hydroxide and the second package comprising guanidine carbonate.

Traditionally, the 2 package compositions are commercially available in liquid, lotion or creamy form in order to facilitate their application onto hair. They generally consist of a cream base in one package and a concentrated solution of guanidinium carbonate in water in the other package. Both are mixed prior to relaxing or straightening the hair in order to have guanidine hydroxide as effective as possible for relaxing the hair.

However, users at home must be very careful with handling of such 2 package compositions as both packages contain alkaline products. Moreover, the two pack systems present challenges to new and existing users on quantity of parts to be mixed per treatment as different hair types, regrowth and hair volume need to be considered before mixing different portions of the parts together. In addition, the use of 2 packs is also disadvantageous for the manufacturers as the manufacturing process is more complex and more errors can occur during packaging and more packaging materials are needed, which is more expensive and ecologically worse.

Therefore, there is a need for hair relaxers, which contain all ingredients including guanidine carbonate in one pack.

U.S. Pat. No. 4,605,018 discloses an anhydrous composition comprising an anhydrous emollient and an effective amount of a water-soluble inorganic oxide. Upon the mixing of an effective amount of water, guanidine hydroxide is formed, which relaxes the hair. According to the examples the composition comprising mineral oil, guanidine carbonate, calcium oxide, stearyl ether, stearyl ether with 2 mol ethylene oxide, Lanolin, trihydroxy stearin and additional several polymers like PQ-10, hydroxymethyl cellulose and bentone gel for viscosity reasons. The relaxer can be obtained by mixing 60 parts of the anhydrous formulation with 40 parts of water.

US 2015/0283041 and 2015/0283042 disclose a powder composition for depilating or for relaxing hair comprising hydroxide-containing compounds, a carbonate compound, starch, a silica material, a liquid fatty substance, an acrylic polymer wax, a sulfur-containing compound and a chelating compound. When the powder is mixed with an aqueous composition, a ready to use hair composition is formed.

Both compositions require polymers to be stable or to have the right viscosity; but polymers should be avoided as far as possible for ecological reasons. Moreover, sulfur-containing compounds have a bed smell. Additionally, powder compositions have the disadvantages of releasing dust.

US2008/025939 refers to hair relaxer formulation comprising i) a derivate of mercaptoacetamide, ii) alkalizing agents like metal hydroxides and/or guanidine carbonate and water. Example 21 discloses a hair straight composition comprising 14 g a derivate of mercaptoacetamide, 0.5 g Guanidine carbonate, 1.5 g metal hydroxide (sodium and lithium), 11 g cetylstearylalcohol (Lanette® O), 2.5 g Cremophor® A20 (cetearylalcohol+20 EO), 3.2 g Vaseline and about 65 g water.

Hair relaxer formulation based on derivates of mercapto smell not very good. Moreover, the disclosed relaxer formulations have a high amount of water and is not a concentrated ready-to use formulation.

WO2016/100714 refers to an agent for shaping hair comprising as a first component a hair treatment composition comprising alkaline hydroxides, carbonate compounds, a cationic polymer, an amphoteric polymer, an organic solvent and water and as a second component an activator. This hair treatment compositions need polymers in high quantities to be stable.

Therefore, there is still a need for a hair relaxer composition, which can be stored as a one-package composition and which is ready to use after mixing with water. Furthermore, the hair relaxer composition should:
be very effective in relaxing
have low-skin irritating properties
have low hair damaging properties
have a very good storage stability
be easily mixable with water at room temperature and
have a good stability without the need of using polymers like acrylic based polymers

After mixing with water the ready-to use hair relaxer composition should:
have a good consistency/viscosity and not run into the eyes
have a good stability
have no bad smell
a long effective time-period for relaxing
provide extra conditioning benefits to the hair

The present invention provides such a beneficial concentrated hair relaxer formulation, which can be stored in 1-pack and which can be easily mixed with water and form a ready-to use hair relaxer composition with the mentioned properties.

In particular, it has now been surprisingly and unexpectedly discovered that a concentrated hair relaxer composition according to the invention comprising Calcium hydroxide and Guanidine Carbonate have excellent storage properties and therefore could be stored in 1-pack.

### DESCRIPTION OF THE INVENTION

One aspect of the invention relates to a concentrated hair relaxer formulation, which is ready to use after mixing with water, comprising
a) 5 to 10 wt % metal hydroxide
b) 5 to 10 wt % guanidine carbonate
c) 50 to 70 wt % emollients
d) 0.01 to 2.0 wt % of a block copolymer of C8-C18 fatty alcohol alkoxylated with ethylene oxide and propylene oxide
e) 0. 5 to 20 wt % O/W emulsifiers,
f) 5 to 15 wt % structurants selected from the group of waxes.

Another aspect relates to a method for producing such a concentrated hair relaxer, a method for relaxing hair, a 1-pack containing such a concentrated hair relaxer and the use of such a 1-pack.

According to the invention the term “hair relaxer composition” or “hair relaxer formulation” means a product, which is ready to use and can put on to hair for relaxing or straightening or smoothing.

The term “concentrated hair relaxer composition” or “concentrated hair relaxer formulation” or “concentrated hair relaxer” or “concentrated relaxer” or short-version “concentrate” means a product, which is ready to use after mixing with water, i.e. after dilution with water.

The terms “compound” or “ingredient” were used in the application in the same way and mean a component of the hair relaxer or of the concentrated hair relaxer.

The terms “1-package” or “1-pack” or “one package” or “one pack” were used in the application in the same way and mean a container which can be closed. It may be a bottle, bag or other kind of container, particularly suitable for liquid formulations.

The term “emollients” means a product, which helps to improve the feel of skin and hair and/or helps to reduce the irritation of the scalp.

The term “wt %” according to the invention is being the weight of the active material and based on the total weight of the concentrated composition.

According to the invention “waxes” are compounds having a melting point above 50° C. and especially above 60° C. The melting point was determined according to ISO 6321.

### Concentrated Hair Relaxer

The inventive concentrated relaxer comprises as an essential ingredient a) at least one metal hydroxide and may be selected from alkali metal hydroxides, alkaline-earth metal hydroxides, and mixtures thereof. Preferred are metal hydroxide selected from the group consisting of sodium hydroxide, potassium hydroxide, lithium hydroxide, calcium hydroxide, magnesium hydroxide, barium hydroxide, strontium hydroxide, and mixtures thereof. Especially preferred is the metal hydroxide a) selected from the group consisting of the calcium hydroxide.

The concentrated relaxer of the present invention comprising a) in an amount ranging from 5 to 10 wt %, preferably from 5 to 8 wt %.

The inventive concentrated relaxer comprises as an essential ingredient b) guanidine carbonate in an amount ranging from 5 to 10 wt %, preferably from 5 to 8 wt %.

The inventive concentrated relaxer comprises as essential ingredient c) emollients in an amount ranging from 50 to 70 wt %, preferably 55 to 70 and especially 55 to 65 wt %.

Emollients can be liquid or waxy-like. According to the invention it is preferred to use one or more liquid emollients or to use one or more liquid emollients in combination with a waxy-like emollient. In general, liquid or waxy-like emollients with good hydrolytic stability can prove to be useful.

Suitable liquid emollients are liquid at 25° C. and can be selected from the group of natural or synthetic sources. Suitable examples of synthetic sources are mineral oil, hydrocarbons or liquid petroleum jelly. Suitable examples of natural sources are fatty alcohols, fatty alcohol esters, fatty acid esters, mono-, and/or di- and/or triglyceride esters, ethoxylated mono-, and/or di- and/or triglyceride esters, guerbet alcohols or ethers. Further suitable emollients are esters of C6-C22 fatty alcohols and/or guerbet alcohols with aromatic carboxylic acids, in particular benzoic acid, esters of C2-C12 dicarboxylic acids with linear or branched alcohols having 1 to 22 carbon atoms or polyols having 2 to 10 carbon atoms and 2 to 6 hydroxyl groups, branched primary alcohols, substituted cyclohexanes, linear and branched C6-C22 fatty alcohol carbonates, such as, for example Dicaprylyl carbonates (Cetiol® CC), Guerbet carbonates based on fatty alcohols with 6 to 18, preferably 8 to 10 C atoms, esters of benzoic acid with linear and/or branched C6-C22 alcohols (e.g. Finsolv TN, Cetiol® AB), linear or branched, symmetrical or asymmetrical dialkyl ethers with 6 to 22 carbon atoms per alkyl group, such as dicaprylyl ether (Cetiol® OE), ring-opening products of epoxidized fatty acid esters with polyols (Cetiol E).

Additional examples of suitable liquid emollients are guerbet alcohols based on fatty alcohols having 6 to 18, preferably 8 to 10, carbon atoms, esters of linear C6-C22 fatty acids with linear or branched C6-C22 fatty alcohols or esters of branched C6-C13 carboxylic acids with linear or branched C6-C22 fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Other suitable liquid emollients are mono-, and/or di- and/or or triglyceride esters based on C6-C18 fatty acids or mono-, and/or di- and/or triglyceride esters based on C6-C18 fatty acids and ethoxylated with 1 to 10 mol ethoxylate, vegetable oils or linear and branched C6-C22 fatty alcohol carbonates. Preferred emollients from the group of mono-, and/or di- and/or triglyceride esters are based on C6-C18 fatty acids and ethoxylated with 1 to 10 mol ethoxylate. Within the ethoxylated fatty acid glycerol esters preference is given to a partial ester of ethoxylated glycerol with a fatty acid mixture derived from coco nut, in particular a mixture of partial esters of ethoxylated glycerol with a fatty acid mixture derived from coconut, wherein the monoester content in the mixture of mono, di- and triester is more than 40% by weight, and it is ethoxylated with 7 mol ethylene oxide. A suitable liquid emollient is the commercial product Cetiol® HE, available by BASF Personal Care Nutrition GmbH.

According to one embodiment the concentrated relaxers comprise liquid emollients, especially mineral oil alone or optional in combination with liquid mono-, and/or di- and/or triglyceride esters based on C6-C18 fatty acids and ethoxylated with 1 to 10 mol ethylene oxide.

According to another embodiment the concentrated relaxers comprise at least one or two liquid emollients and a waxy-like emollient. Examples for a waxy-like emollient are waxes with a melting point between 30° C. and 50° C., selected from the group of natural based-waxes. Typical examples of are candelilla wax, carnauba wax, Japan wax, espartograss wax, cork wax, guaruma wax, rice oil wax, sugar cane wax, ouricury wax, beeswax, shellac wax, spermaceti, lanolin (wool wax), uropygial fat, ceresine, ozocerite (earth wax), petrolatum, hydrogenated jojoba waxes Butyrospermum Parkii (Shea) Butter, like Cetiol® SB 45. Preferred as a waxy-like emollient is Butyrospermum Parkii (Shea) Butter.

According to an especially preferred embodiment of the invention the concentrated relaxer comprising as emollients c) a combination of the liquid emollients selected from the group consisting of mineral oil and a liquid mono-, and/or di- and/or triglyceride ester based on C6-C18 fatty acids and ethoxylated with 1 to 10 mol ethoxylate and the waxy-like emollient Butyrospermum Parkii (Shea) Butter.

According to the especially preferred embodiment the concentrated relaxer comprising as emollients c) a combination of 50 wt % to 63 wt % mineral oil and 0.5 to 2 wt % liquid mono-, and/or di- and/or triglyceride esters based on C6-C18 fatty acids and ethoxylated with 1 to 10 mol ethylene oxide and 1 to 5.0 wt % waxy-like emollient Butyrospermum Parkii (Shea) Butter—wt % based on concentrated relaxer.

The inventive concentrated relaxers comprise additional as an ingredient d) 0.01 to 2 wt % of a block copolymer of C8-C18 fatty alcohol alkoxylated with ethylene oxide and propylene oxide, preferably alkoxylated with 5 to 15 mol ethylene oxide and 1 to 3 propylene oxide, and especially lauryl alcohol alkoxylated with 9 mol ethylene oxide and 1 mol propylene oxide (so-called PPG-PEG-9 Lauryl Glycol ether or PPG-PEG-9 Lauryl Glycol).

Without being bound to a theory, it is assumed that the compound d) acts as a so-called solubilizer, i.e. causes another compound to be more soluble in another compound. In the present invention, it is believed that d) acts as a solubilizer, presumably enhances the solubility of the waxes described as structurants f) in the emollient c). In any case, a better storage stability is observed due to the presence of d).

According to one preferred embodiment of the present invention d) is amixture (=combination) of 0.01 to 1.5 wt % polyethoxylated hydrogenated castor oil, ethoxylated with 20 to 100 mol ethylene oxide, and 0.01 to 2 wt % of a block copolymer of C8-C18 fatty alcohol alkoxylated with ethylene oxide and propylene oxide, especially a mixture of 0.01 to 1.5 wt % polyethoxylated hydrogenated castor oil, ethoxylated with 30 to 50 mol ethylene oxide, and a block copolymer of C8-C18 fatty alcohol alkoxylated with 5 to 15 mol ethylene oxide and 1 to 3 propylene oxide.

Castor oil (CAS-Nr. 8001-79-4) is a vegetable oil and contains glycerides, especially triglycerides of fatty acids having C10 to C22 alkyl or alkenyl moieties which incorporate a hydroxyl group. Hydrogenation of castor oil produces hydrogenated castor oil by converting double bonds, which are present in the starting oil as ricinoleyl moieties. These moieties are converted to ricinoleyl moieties, which are saturated hydroxyalkyl moieties, e.g. hydroxystearyl.

The hydrogenated castor oil (HCO) may be processed in any suitable starting form, including, but not limited those selected from solid, molten and mixtures thereof. Useful hydrogenated castor oil (HCO) may have the following characteristics: more than 80 wt %, especially 80-90 wt % triglycerides of ricinoleic acid. The residue may be free fatty acids, water and other impurities. The most preferred hydrogenated castor oil is ethoxylated with 30 to 50 mol ethylene oxide.

Very excellent properties were observed, when d) is a combination of three compounds and contain additionally as a third component C12-C18 fatty alcohols ethoxylated with 1 to 10 mol ethylene oxide, especially 7 mol ethylene oxide.

The inventive concentrated hair relaxers comprise as d) in particular a mixture of
0.01 to 1.5 wt % polyethoxylated hydrogenated castor oil ethoxylated with 20 to 100 mol ethylene oxide and
0.01 to 2.0 wt % a block copolymer of C8-C18 fatty alcohol alkoxylated with ethylene oxide and propylene oxide, especially with 5 to 15 mol ethylene oxide and 1 to 3 propylene oxide and more preferable a mixture of
0.01 to 1.5 wt % polyethoxylated hydrogenated castor oil ethoxylated with 20 to 100 mol ethy-lene oxide, preferable ethoxylated with 30 to 50 mol ethylene oxide and
0.01 to 2.0 wt % block copolymer of C8-C18 fatty alcohol alkoxylated with ethylene oxide and propylene oxide, preferable with 5 to 15 mol ethylene oxide and 1 to 3 propylene oxide and
0.01 to 2.0 wt % C12-C18 fatty alcohols ethoxylated with 1 to 10 mol ethylene oxide, especially 7 mol ethylene oxide.

The preferred mixture with three compounds is for example commercially available as a premix, e.g. under the Trademark Eumulgin® HPS, BASF. In case such a premix is used, it is preferred to use it in amounts of 1.0 to 3.5 wt %—based on the concentrated hair relaxers.

The inventive concentrated relaxers comprise additional O/W emulsifier as component e) in amounts of 0.5 to 20 wt %. O/W emulsifiers are needed for o/w emulsions, this means an oil is dispersed in a continuous water phase. O/W emulsifiers have in general an HLB (Hydrophilic-Lipophilic Balance) between 8 and 16. According to the invention the concentrated relaxer seems to need emulsifiers as the relaxer concentrates have to be mixed with water to make them ready for use as a relaxer formulation.

According to the invention suitable O/W emulsifiers are selected from the group consisting of:
C12/C18 fatty alcohols
C12/C18 fatty alcohols ethoxylated with 15 to 40 mol ethylene oxide
C12/C18 fatty alcohol ethoxylated with 2 to 30 mol ethylene oxide and 1 to 5 mol propylene oxide
C16/C18 fatty acids

C16/C18 fatty acids ethoxylated with 40 to 150 mol ethylene oxide
castor oil and/or hardened castor oil, optional ethoxylated with 1 to 15 mol ethylene oxide
partial esters of glycerol and/or sorbitan esterfied with fatty acids with 12 to 22 carbon atoms and/or hydroxycarboxylic acids with 3 to 18 carbon atoms and optional ethoxylated with 1 to 30 moles of ethylene oxide
partial esters of polyglycerol (average degree of auto-condensation 2 to 8), polyethylene glycol (molecular weight 400 to 5000), trimethylolpropane, pentaerythritol, sugar alcohols (e.g. sorbitol), alkyl glucosides (e.g. methyl glucoside, butyl glucoside, butyl glucoside, butyl glucoside, butyl glucoside, butyl glucoside, butyl glucoside, butyl glucoside, butyl glucoside) Methyl glucoside, butyl glucoside, lauryl glucoside) and poly glucosides (e.g. cellulose) with fatty acids containing 12 to 22 carbon atoms and/or hydroxycarboxylic acids containing 3 to 18 carbon atoms and optional ethoxylated thereof with 1 to 30 mol of ethylene oxide
mixed esters of pentaerythritol, fatty acids, citric acid and fatty alcohol and/or mixed esters of fatty acids containing 6 to 22 carbon atoms, methylglucose and polyols like glycerol or polyglycerol.

Examples hydroxystearic acid monoglyceride, hydroxystearic acid diglyceride, isostearic acid monoglyceride, stearic acid monoglyceride, oleic acid monoglyceride, and/or optional ethoxylated with 1 to 30, preferably 5 to 10, moles of ethylene oxide. to products of 1 to 30, preferably 5 to 10, moles of ethylene oxide. Other examples are sorbitan esters like sorbitan monoisostearate, sorbitan mono stearate, sorbitan diisostearate, sorbitan triisostearate, sorbitan monooleate, sorbitan sesquioleate, sorbitan diolate, sorbitan trioleate, sorbitan triricinoleate, sorbitan mono citrate and/or ethoxylated with 1 to 30, preferably 5 to 10, moles of ethylene oxide to. Other examples are polyglycerol esters like polyglyceryl-2 dipolyhydroxystearates (Dehymuls® PGPH, commercially available from BASF), polyglycerol-3-diisostearates (Lameform TGI®, commercially available from BASF), Polyglyceryl-4 Isostearate, Polyglyceryl-3 Oleate, Trimethylpropane-EO/PO-trioleate.

Other suitable examples are partial esters of glycerides such as lauric acid monoglyceride, coconut fatty acid monoglyceride, palmitic acid monoglyceride, stearic acid monoglyceride (also named as Glyceryl mono stearate or glyceryl monostearate), tallow fatty acid monoglyceride as well as their technical mixtures, which may still contain small amounts of di- and or triglyceride subordinated from the manufacturing process.

Examples of fatty acids are palmitic and stearic acid or their technical mixtures as well as their adducts with ethylene oxide, especially polyethoxylated stearate with 70 to 120 mol ethylene oxide. Polyethoxylated stearate with 70 to 120 mol ethylene oxide is also named as PEG-70-120 Stearate. Particularly, PEG-100 Stearate is preferred.

According to an embodiment according to the invention concentrated relaxer are preferred comprising at least one O/W emulsifier mixture and preferred comprising two O/W emulsifier mixtures.

One useful O/W emulsifier mixture is a mixture of a partial ester of glycerol and a polyethoxylated fatty acid, especially Glyceryl monostearate (or named stearic acid monoglyceride) and C18 fatty acids ethoxylated with about 100 mol ethylene oxide (also named PEG-100 Stearate). An example of such a O/W emulsifier mixture is Emulgade® 165, available from BASF.

Another useful O/W emulsifier mixture is a mixture of a C12-18 fatty alcohol, especially C16/C18 fatty alcohol, and an C12-18 fatty alcohol, especially an C16/C18 fatty alcohol, ethoxylated with 15-40 mol ethylene oxide, especially ethoxylated with 30 mol ethylene oxide. An example of such a mixture of C16/18 fatty alcohol and C16/18 fatty alcohol ethoxylated with 30 mol ethylene oxide is Lanette® Wax AO, available from BASF.

According to the most preferred embodiment of the invention the concentrated relaxer comprising both preferred O/W emulsifier mixtures and especially 0.1 to 5 wt % O/W emulsifier mixture of a partial ester of glycerol and a polyethoxylated fatty acid, especially Glyceryl monostearate (or named stearic acid monoglyceride) and C18 fatty acids ethoxylated with about 100 mol ethy-lene oxide and 0.4 to 15 wt % O/W emulsifier mixture of a C12-18 fatty alcohol, especially C16/C18 fatty alcohol, and an C12-18 fatty alcohol, especially C16/C18 fatty alcohol, ethoxyla-ted with 15-40 mol ethylene oxide, especially 30 mol ethylene oxide.

The inventive concentrated relaxers comprise additional 5 to 15 wt % structurants f) selected from the group of waxes. Without being bound to a theory, it is assumed that the structurants acts like consistency agents and thicken the concentrated hair relaxer. At least the presence of structurants is very useful as they improve the storage stability of the inventive concentrated relaxers and the ready-to-us formulation.

Preferred structurants f) are waxes selected from the group consisting of natural waxes, especially from the group consisting of fatty waxes. Preferred are waxes of higher fatty alcohols like cetyl alcohol, stearyl alcohol and behenyl alcohol. Especially preferred is behenyl alcohol, commercially available as Lanette® 22.

For the storage properties of the concentrated relaxer it is advantageous that the concentrated relaxers comprise less than 5 wt % water, especially 0 to 3 wt % water, and in particular to be water-free.

According to the invention the inventive concentrated hair relaxer may contain additional ingredients like thickeners, humectants, preservatives, accelerator, complexing agents, nonionic surfactants and/or anionic surfactants. Examples for anionic surfactants are fatty alcohol sulfates types such as e.g. sodium cetearyl sulfate, sodium stearyl sulfate or sodium stearoyl glutamates, and/or examples for nonionic surfactants are alkyl polyglycosides. It is possible to replace humectants like glycerol in the afore mentioned concentrate by polyols like sugars such as glucose, sorbitol or by natural products such as honey. Useful complexing agents are EDTA (ethylene-diaminetetraacetic acid), NTA (nitrilotriacetic acid), HEDP (1-hydroxyethane (1,1-di-phosphonic acid)) and/or DTPA (diethylenetriaminepentaacetic acid. In an embodiment, the acrylic polymer If desired thickeners can be contained like natural thickener as hydroxypropylmethylcellulose, hydroxyethylcellulose or synthetic thickeners like crosslinked acrylic polymers like sodium polyacrylate, carbomer, acrylates C10-30 alkyl acrylate crosspolymer, and mixtures thereof. According to the invention synthetic thickeners are not necessary and are therefore not present.

According to the invention the inventive concentrated hair relaxer may contain additional ingredients like cationic polymers as deposition agents, i.e. by using them the deposition of the compounds on hair was improved and give them a pleasant and a soft feeling. These cationic deposition polymers can include at least one of a cationic guar polymer, a cationic non-guar galactomannan polymer, a cationic tapioca polymer, a cationic copolymer of acrylamide monomers and cationic monomers, and/or a synthetic, non-crosslinked, cationic polymer.

However, it is preferred that the hair relaxer formulation according to the invention are comprising less than 5 wt % polymers based on (meth)acrylic acid or their derivates, especially 0 wt %.

Additionally, silicone compounds can be present, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resinlike at room temperature. Other suitable silicone compounds are simethicones which are mixtures of dimethicones with an average chain length of 200 to 300 dimethylsiloxane units and hydrogenated silicates.

Additionally, film formers may be present. Customary film formers are, for example, chitosan, microcrystalline chitosan, quaternized chitosan, polyvinylpyrrolidone, vinylpyrrolidone-vinyl acetate copolymers, polymers of the acrylic acid series, quaternary cellulose derivatives, collagen, hyaluronic acid and salts thereof and similar compounds.

If desired, further protein hydrolysates known from the prior art may be used, for example based on keratin such as the commercially available Nutrilan® Keratin W PP, or based on wheat, such as Gluadin® WLM Benz, Gluadin® WK or Gluadin® WP. It is also possible to add small amounts of free amino acids such as lysine or arginine.

Advantageously the claimed concentrated relaxer formulation does not need sulfur containing compounds and therefore it is preferred that the claimed hair relaxer formulation comprising less than 5 wt % sulfur containing compounds, especially 0 wt %.

The great advantage of the present invention is that the concentrated relaxer can be filled and stored in one package without causing undesirable reactions of the components which reduce the reactivity of the alkaline compounds. Accordingly, another object of the present invention is directed to a hair relaxer formulation according to claim 1, filled in 1-pack.

Particularly preferably, the 1-pack (one-package) are sealable bottles or bags, in particular plastic bottles or also plastic bags which are additionally coated. After filling the concentrated relaxer according to the invention, the bottles or bags are sealed in a conventional manner known to the skilled person. The user can then open these packages before mixing with water. Generally, about amounts of 50 to 100 grams of relaxer are placed in the package. It is also possible to put the concentrated relaxers in a multiple-use packaging, e.g. of up to 2 kg that helps to economise the use of packaging material.

Another object of the present invention relates to a method for producing a concentrated hair relaxer formulation according to claim 1, wherein
at least 50 wt % emollients c) were submitted in the reactor vessel and
one after the other were added and mixed in the following order a), b) and optional further emollients c), O/W emulsifier e), component(s) d) and structurant f) and
all mixed components were heated up to 70° C. and stirred for about 5 to 15 minutes, and
cooled down.

It is preferred to submit the emollient, e.g. mineral oil, in the reaction vessel and to add and mix all other ingredients in the following order: a), b), optional further c), e), d) and f). After adding and mixing all mixed components were heated up to 70° C., preferred to 65 to 70° C. In this temperature range the ingredients are a molted mix and can be stirred for about 5 to 15, preferred 10 to 15 minutes. It is advantageous not to raise the temperature above 75° C., preferred 72° C.

After stirring, the mixture is allowed to cool down; it is preferred to fill the cooled down mixture in the 1-pack, in particular at about 30° C.

According to the invention the concentrated relaxer will be mixed before it is ready to use. Therefore, another object of the invention is a method for relaxing hair, wherein the concentrated hair relaxer formulation according to claim 1 were mixed with water in a ration 3.1 to 4.1 and stirred until it is ready to use.

It is preferred to mix 75 parts of the concentrated relaxer with 25 parts water; in general, a thick emulsion was obtained, which can be easily put on the scalp. The water may or may not be demineralized. Preferably, the water has a temperature that corresponds to the room temperature.

Further object of the present invention relates to 1-pack containing a concentrated hair relaxer formulation comprising
a) 5 to 10 wt % metal hydroxide
b) 5 to 10 wt % guanidine carbonate
c) 50 to 70 wt % emollients
d) 0.01 to 2.0 wt % of a block copolymer of C8-C18 fatty alcohol alkoxylated with ethylene oxide and propylene oxide
e) 0.5 to 20 wt % O/W emulsifiers
f) 5 to 15 wt % structurants selected from the group of waxes.

And a further object relates to the use of a 1-pack defined in claim 18 after mixing with water as a ready-to use hair relaxer formulation.

### EXAMPLES

### A) Concentrated Hair Relaxer

### Example 1: Concentrated hair relaxer

The concentrated relaxer was prepared by submitting 56 wt % mineral oil in the reactor vessel and adding and mixing all ingredients in the given order to the preparation vessel:

7.50 wt % Calcium hydroxide
7.50 wt % Guanidine Carbonate
3.00 wt % Butyrospektum Parkii Butter (Emollient; component c))
3.00 wt % Mixture of Glyceryl mono stearate and PEG-100 stearate (O/W emulsifier; component e) Emulgade® 165)
1.00 wt % PEG-7 Glycerylcocoate (emollient; component c) Cetiol® HE)
2.00 wt % Premix PEG-40 hydrogenated Castor Oil and Coceth-7 and PPG-PEG-9 Lauryl Glycol Ether (component d); Eumulgin® HPS)
10.0 wt % Mixture of Cetearyl Alcohol and Ceteareth-30 (O/W emulsifier; component e) Lanette® Wax AO)
10.0 wt % Behenyl Alcohol (Structurant; component f); Lanette® 22)

Afterwards all ingredients were heated to 65 to 70° C. When molten all ingredients were stirred for about 10 minutes. Then the mixture was cooled down. During the process contact with water was avoided. The concentrated relaxer was filled in a bottle and closed.

### Example 2

Example 1 was repeated but with a different component d).

2 wt % of the premix Eumulgin® HPS was replaced by

1.0 wt % PEG-40 hydrogenated Castor Oil and 1.0 wt % PPG-PEG-9 Lauryl Glycol ether

### Example 3

Example 1 was repeated but with a different component d).

2 wt % of the premix Eumulgin® HPS was replaced by

0.5 wt % PEG-40 hydrogenated Castor Oil and 1.5 wt % PPG-PEG-9 Lauryl Glycol ether.

### Example 4

Example 1 was repeated, but with a different component d). 2 wt % of the premix Eumulgin® HPS was replaced by 2 wt % PPG-PEG-9 Lauryl Glycol Ether (component d).

Results: All four concentrated hair relaxer are very stable. After 1 month no segregation could be watched.

### Comparison Example 5

Example 1 was repeated, but with a different component d). 2 wt % of the premix Eumulgin® HPS was replaced by 2 wt % PEG-40 hydrogenated Castor Oil.

Result: The concentrated hair relaxer is unstable and cannot be taken any further.

### B) Ready-to Use Hair Relaxer Formulation

### Example 6

25 parts water was added to 75 parts of the concentrated hair relaxer according example 1 to 4. A thick emulsion was formed in situ-, which is ready to use for applying.

The ready-to use hair relaxer formulation comprising Example 1 to 4 were very stable, and no bad smell occurred within applying and relaxing time.

The ready-to use hair relaxer formulation comprising Example 1 was applied to strands of curly Brasilian hair and left for 30 minutes. After that the relaxer formulation was washed out and the hair shampooed and dried. The achieved hair relaxion was measured as hair extension by measuring the strand length before and after using the relaxer formulation and given in %: 120.

1. A concentrated hair relaxer formulation, which is ready to use after mixing with water, comprising
a) 5 to 10 wt % of a metal hydroxide,
b) 5 to 10 wt % guanidine carbonate,
c) 50 to 70 wt % of an emollient,
d) 0.01 to 2.0 wt % of a block copolymer of C8-C18 fatty alcohol alkoxylated with ethylene oxide and propylene oxide,
e) 0.5 to 20 wt % of an O/W emulsifier,
f) 5 to 15 wt % of a structurant comprising a wax.

2. The hair relaxer formulation according to claim 1, wherein the metal hydroxide a) comprises calcium hydroxide.

3. The hair relaxer formulation according to claim 1, wherein the emollient c) is a combination of liquid emollients selected from the group consisting of mineral oil, liquid mono- and/or di- and/or triglyceride esters based on C6-C18 fatty acids and ethoxylated with 1 to 10 mol ethylene oxide, and Butyrospermum Parkii (Shea) Butter.

4. The hair relaxer formulation according to claim 1, wherein the block copolymer of C8-C18 fatty alcohol according to d) is alkoxylated with 5 to 15 mol ethylene oxide and 1 to 3 mol propylene oxide.

5. The hair relaxer formulation according to claim 1, wherein d) is a mixture of 0.01 to 1.5 wt % polyethoxylated hydrogenated castor oil ethoxylated with 20 to 100 mol ethylene oxide, and 0.01 to 2 wt % of a block copolymer of C8-C18 fatty alcohol alkoxylated with ethylene oxide and propylene oxide.

6. The hair relaxer formulation according to claim 1, wherein d) is a mixture of
0.01 to 1.5 wt % polyethoxylated hydrogenated castor oil ethoxylated with 20 to 100 mol ethylene oxide, and
0.01 to 2.0 wt % block copolymer of C8-C18 fatty alcohol alkoxylated with ethylene oxide and propylene oxide, and
0.01 to 2.0 wt % of a C12-C18 fatty alcohol ethoxylated with 1 to 10 mol ethylene oxide.

7. The hair relaxer formulation according to claim 1, wherein the O/W emulsifier e) is selected from the group consisting of C12/C18 fatty alcohols, C12/C18 fatty alcohols ethoxylated with 15 to 40 mol ethylene oxide, C12/C18 fatty alcohol ethoxylated with 2 to 30 mol ethylene oxide and 1 to 5 mol propylene oxide, C16/C18 fatty acids, C16/C18 fatty acids ethoxylated with 40 to 150 mol ethylene oxide, castor oil and/or hardened castor oil, optional ethoxylated with 1 to 15 mol ethylene oxide, partial esters of glycerol and/or sorbitan esterfied with fatty acids with 12 to 22 carbon atoms and/or hydroxycarboxylic acids with 3 to 18 carbon atoms and optional ethoxylated with 1 to 30 moles of ethylene oxide, partial esters of polyglycerol (average degree of auto-condensation 2 to 8), polyethylene glycol (molecular weight 400 to 5000), trimethylolpropane, pentaerythritol, sugar alcohols, alkyl glucosides methyl glucoside, butyl glucoside, lauryl glucoside, polyglucosides with fatty acids containing 12 to 22 carbon atoms and/or hydroxycarboxylic acids containing 3 to 18 carbon atoms and optional ethoxylated thereof with 1 to 30 mol of ethylene oxide; mixed esters of pentaerythritol, fatty acids, citric acid, fatty alcohol and/or mixed esters of fatty acids containing 6 to 22 carbon atoms, methylglucose, polyols, glyceryl monostearate, polyethoxylated stearate with 70 to 120 mol ethylene oxide, C12-C18 fatty alcohol, and polyethoxylated C12-C18 fatty alcohol with 15 to 40 mol ethylene oxide.

8. The hair relaxer formulation according to claim 1, wherein the O/W emulsifier e) comprises a mixture of glyceryl mono stearate and C18 fatty acid ethoxylated with about 100 mol ethylene oxide.

9. The hair relaxer formulation according to claim 1, wherein the O/W emulsifier e) comprises a mixture of C12-C18 fatty alcohol and C12-C18 fatty alcohol ethoxylated with 15 to 40 mol ethylene oxide.

10. (canceled)

11. (canceled)

12. The hair relaxer formulation according to claim 1, wherein structurant f) is a natural.

13. The hair relaxer formulation according to claim 1 comprising less than 5 wt % water.

14. The hair relaxer formulation according to claim 1, wherein the formulation comprises less than 5 wt % sulfur containing compounds.

15. The hair relaxer formulation according to claim 1, wherein the formulation comprises less than 5 wt % polymers based on (meth)acrylic acid or their derivates.

16. The hair relaxer formulation according to claim 1, filled in a 1-pack.

17. A method for producing a concentrated hair relaxer formulation according to claim 1, wherein
at least 50 wt % emollient c) is added in a reactor vessel and
one after the other is added and mixed in the following order a), b), and optional further emollient c), O/W emulsifier e), component d), and structurant f), and
all mixed components are heated up to 70° C. and stirred for about 5 to 15 minutes, and
cooled down.

18. The method for producing a concentrated hair relaxer formulation according to claim 17, wherein the cooled down mixture is filled in a 1-pack.

19. A method for relaxing hair, wherein the concentrated hair relaxer formulation according to claim 1 is mixed with water in a ratio 3:1 to 4:1, stirred until it is ready to use, then applied to the hair.

20. A 1-pack containing a concentrated hair relaxer formulation comprising
a) 5 to 10 wt % of a metal hydroxide,
b) 5 to 10 wt % guanidine carbonate,
c) 50 to 70 wt % of an emollient,
d) 0.01 to 2.0 wt % of a block copolymer of C8-C18 fatty alcohol alkoxylated with ethylene oxide and propylene oxide,
e) 0.5 to 20 wt % of an O/W emulsifier,
f) 5 to 15 wt % of a structurant comprising a wax.

21. (canceled)

22. The hair relaxer formulation according to claim 5, wherein d) is a mixture of 0.01 to 1.5 wt % polyethoxylated castor oil, ethoxylated with 30 to 50 mol ethylene oxide, and 0.01 to 2 wt % of a block copolymer of a C8 18 fatty alcohol, alkoxylated with 5 to 15 mol ethylene oxide and 1 to 3 mol propylene oxide.

23. The hair relaxer formulation according to claim 1, wherein the relaxer formulation comprises a mixture of
glyceryl mono stearate and C18 fatty acid ethoxylated with about 100 mol ethylene oxide, and
C12-18 fatty alcohol and C12-18 fatty alcohol ethoxylated with 15 to 40 mol ethylene oxide.

24. a hair relaxer formulation according to claim 1, wherein the relaxer formulation comprises 0.1 to 5 wt % of a mixture of glyceryl mono stearate and C18 fatty acid ethoxylated with about 100 mol ethylene oxide, and 0.4 to 15 wt % of a mixture of C12-18 fatty alcohol and C12-18 fatty alcohol ethoxylated with 15 to 40 mol ethylene oxide.
